(19) United States
(12) Patent Application Publication
(10) Pub. No.: US 2007/0299538 A1
(43) Pub. Date: Dec. 27, 2007

(54) Ease of use tissue repair patch

(76) Inventor: Peter J. Roeber, Wallingford, PA (US)

Correspondence Address: GORE ENTERPRISE HOLDINGS, INC., 551 PAPER MILL ROAD, P. O. BOX 9206, NEWARK, DE, 19714-9206, US

(21) Appl. No.: 11/474,673

(22) Filed: Jun. 26, 2006

(51) Int. Cl. A61F 2/02

(52) U.S. Cl. 623/23.72

(57) ABSTRACT

An implantable medical device is disclosed having a tissue repair material with two sides and an outer perimeter and a cuff formed from the outer perimeter to overlap onto a side of the tissue repair material. The device may be used with a removable support member.

### BACKGROUND OF THE INVENTION

Surgically implanted patches are used to treat wall defects, such as hernia, umbilical repairs and other such defects. The patches are typically implanted and remain in a patient. Several patents and/or published patent applications were identified for hernia patches comprising two layers of biocompatible mesh with one or more pouches, pockets or slits for insertion of either a surgeon's finger or a trocar or other fastening and/or positioning tool to aid in implanting the hernia patch.

U.S. Pat. Nos. 5,634,931, 5,769,864, and 5,916,225 by Kugel which disclose hernia patches comprising a first layer of inert mesh material selectively sized and shaped to extend across and beyond a hernia, a second layer of inert synthetic mesh material which overlies the first layer, wherein the first and second layers are joined together to define a periphery of a pouch between the two layers, and a resilient monofilament loop located within the pouch to urge the patch to conform to a planar configuration. An access slit is formed in one of the layers for insertion of the surgeon's finger into the pouch to facilitate insertion. U.S. Pat. Nos. 6,176,863 and 6,280,453 by Kugel et al. disclose similar hernia mesh patches with a single slit for insertion of the surgeon's finger.

U.S. Pat. No. 6,174,320 by Kugel et al. discloses a hernia patch with an additional slit through both layers of the mesh material and terminating in an enlarged opening for placement around a patient's chord structure. This hernia patch is used in repair of inguinal hernias.

U.S. Publication No. 2004/0215219 by Eldridge et al. discloses an implantable prosthesis for hernia repair comprising two layers of material that permit the formation of adhesion with tissue or muscle, at least one pocket, more preferably two pockets formed between the first and second layers for insertion of a surgeon's fingers and a layer of barrier material resistant to formation of adhesions with tissue or muscle attached to the second layer at discrete locations.

In addition, U.S. Pat. No. 5,922,026 by Chin discloses a prosthetic strip or patch which includes pockets at each end into which a fastener tool can be inserted and used to position and secure the patch to tissues and ligaments in the body.

There is a strong need in the art for a flexible tissue repair patch which allows for accuracy and ease in delivery and placement by a surgeon. It is also desired that the patch provides improved protection of the surrounding tissues during procedures such as hernia repairs.

The present invention fills the need in that it is different from the slit and finger fitting pockets described in the above hernia patches. Additionally, the present invention allows for fixation of the patch at the outer most perimeter of the implanted patch.

### SUMMARY OF THE INVENTION

An implantable medical device is provided comprising a tissue repair material having two sides and an outer perimeter with at least one side adapted for ingrowth of cells, and a cuff formed from the outer perimeter to overlap onto a side of the tissue repair material. A removable support member may be removably positioned under the cuff for ease of use manipulation during implant procedures.

### DESCRIPTION OF THE DRAWINGS

FIG. 1 illustrates a perspective view of the present invention.

FIG. 2 illustrates an exploded view of the present invention including a removable support member.

FIG. 3A illustrates a top plan view of the present invention.

FIG. 3B illustrates a cross-sectional view of the present invention showing how the cuff retains the removable support member.

FIG. 3C illustrates a detailed section view of the mesh material with the formed cuff retaining the removable support member.

FIG. 4A illustrates a mesh material constructed from a composite material, having more than one continuous layer.

FIG. 4B illustrates a mesh material constructed from a non-continuous composite material, having more than one layer.

FIG. 4C illustrates a mesh material constructed from more than one material.

FIG. 5A illustrates a bottom plan view of the mesh prior to cuff forming showing the fold line and the section of bottom surface area that has been roughened.

FIG. 5B illustrates a cross-sectional view of the mesh prior to cuff forming showing the top and bottom surface areas that have been roughened.

FIG. 5C illustrates a cross-sectional view of the device after cuff formation showing the orientation of the surfaces that have been roughened.

FIG. 5D illustrates a cross-sectional view of the device after cuff formation showing the orientation of the surfaces that have been roughened.

FIG. 6A illustrates a top plan view of the present invention.

FIG. 6B illustrates a top plan view of the present invention showing an alternative cuff form.

FIG. 6C illustrates a cross-sectional view of the mesh showing a continuous cuff.

FIG. 6D illustrates a cross-sectional view of the mesh showing a non-continuous cuff.

FIG. 7 illustrates a top plan view of various removable support member designs showing that the support member can have a continuous boundary, a slit boundary, a boundary with relief cuts as well as different access hole configurations.

FIG. 8A illustrates a cross-sectional view of the removable support member showing an edge reinforcement.

FIG. 8B illustrates a cross-sectional view of the removable support member showing a non-planar configuration.

FIG. 8C illustrates a cross-sectional view of the removable support member showing internal reinforcement.

FIG. 8D illustrates a cross-sectional view of the removable support member showing a non-continuous configuration.

FIG. 8E illustrates a cross-sectional view of the removable support member showing a peripheral ring only configuration.

FIG. 9A illustrates a top plan view of a mesh during manufacture showing a pleated cuff configuration.

FIG. 9B illustrates a cross-sectional view of the mesh during manufacture showing a pleat construction.

FIG. 10 illustrates a top plan view of various shapes and configurations of the present invention.

FIG. 11A illustrates a top plan view of an alternative removable support member.

FIG. 11B illustrates a cross-sectional view of an alternative removable support member and the direction of removal.

FIG. 12 illustrates a top plan view of a non-continuous cuff showing two segments.

FIG. 13A illustrates a top plan view of a non-continuous cuff showing four segments.

FIG. 13B illustrates a cross-sectional view of a non-continuous cuff showing how the segment retains the removable support member.

FIG. 14 illustrates a cross-sectional view of a two-layer mesh material with the bottom layer extending beyond the cuff.

FIG. 15 illustrates a cross-sectional view of a device with a first cuff and a second cuff formed by folding the first cuff over onto the first cuff.

### DETAILED DESCRIPTION OF THE INVENTION

The present invention is an implantable device designed to repair damaged tissue such as soft tissue defects, wall defects, hernia, umbilical repairs and other such defects. The device is formed of a planar tissue repair material having two sides and an outer perimeter, and a cuff formed from the outer perimeter of the tissue repair material to overlap onto a side of the tissue repair material. The present invention may further include a removable support member over which the tissue repair material is overlapped to cover at least a portion of the removable support member.

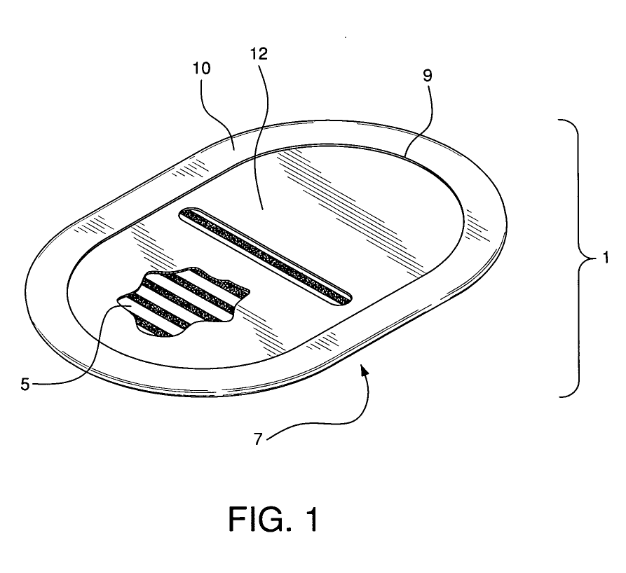

As shown in FIG. 1, an implantable medical device 1 is formed from a planar sheet of tissue repair material 3. The tissue repair material 3 has two sides shown as top side 5 and bottom side 7 and an outer perimeter 9. A cuff 10 is shown formed from the tissue repair material 3 to overlap onto a side of the tissue repair material 3. The cuff 10 allows a fixation area 18 to be formed on the cuff 10, and in particular at the outer edge 20 of the implantable medical device 1. A removal support member 12 fits under the cuff for implantation ease.

The tissue repair material 3 may be any material suitable for medical use, for instance, polymeric materials including polytetrafluoroethylenes and expanded polytetrafluoroethylenes.

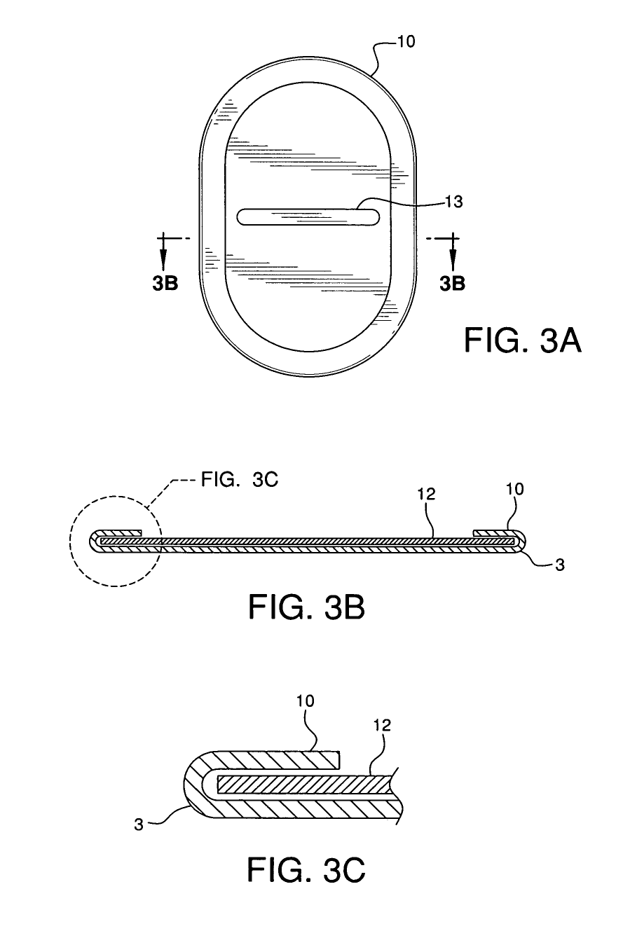

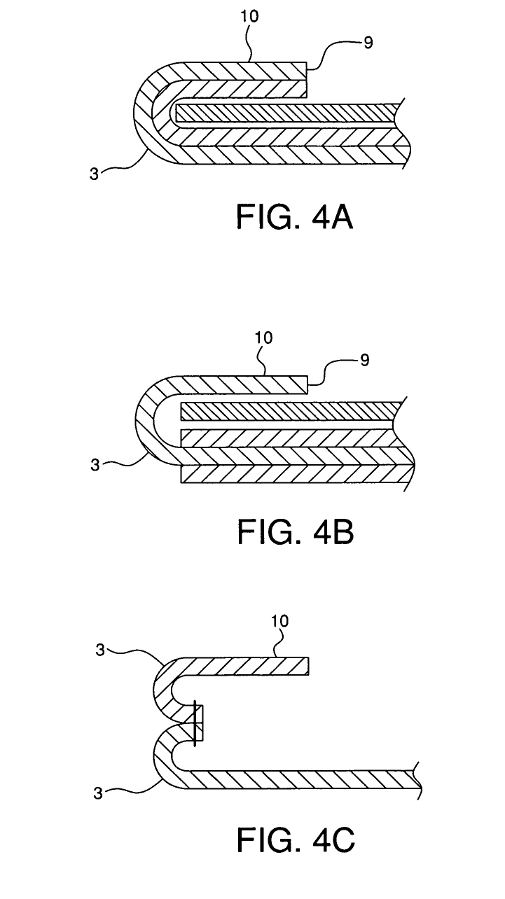

While the tissue repair material 3 is shown as having a smooth outer perimeter, the invention is not limited to this configuration. It is anticipated that the outer perimeter 9 of the tissue repair material 3 could be smooth, irregular, patterned, notched or other shaped variations. It is further contemplated that tissue repair material 3 may comprise variations consistent with the embodied invention, depending on the application for which it is intended. For instance, the tissue repair material 3 may be comprised of a single material composition, as shown in FIGS. 3A through 3C; or may comprise two or more materials, as shown in FIG. 4A as a composite material having more than one continuous layer. FIG. 4B further illustrates that tissue repair material 3 can be constructed from a non-continuous composite material, having more than one layer. The tissue repair material 3 is preferred to be in the form of a single piece of material which achieves the overlap making the cuff 10, by folding the edges of the outer perimeter 9 of the tissue repair material 3 onto a side. However, it is readily apparent that more than one piece of tissue repair material 3 may be used with the device, as shown in FIG. 4C. FIG. 4C shows the use of a single piece of material which forms the bottom side joined to at least one piece of material to form the cuff 10. The pieces may be joined at their perimeter, by sewing, ultrasonic welding, bonding, or other suitable methods to form a device with two sides and an outer perimeter.

The tissue repair material 3 may be in the form of a sheet, mesh, web, wovens, non-wovens, knits, or any other suitable material. In one embodiment the implantable device of the present invention is comprised of a tissue repair material 3 that is an infection resistant mesh material. In this embodiment the tissue repair material 3 comprises one or more matrix-forming polymers, such as biomedical polyurethanes, biomedical silicones and biodegradable polymers and antimicrobial agents, and combinations of matrix-forming polymers with therapeutic agents such as silver salts and/or chlorhexidine or its salts. The matrix may be coated with an antimicrobial agent in a solvent solution, causing the coating material to enter into the interstices and dry, resulting in an infection resistant matrix.

The tissue repair material 3 may further have one or both sides adapted to allow an ingrowth of cells. In another aspect, the tissue repair material 3 may comprise at least one bioabsorbable material. The biosorbable material may be present throughout the tissue repair material 3 or may exist on designated areas only.

The cuff 10 formed on the device is continuous with and integral to the tissue repair material 3. The cuff 10 and tissue repair material 3 may be formed of substantially the same materials, or substantially different materials depending upon the desired application of the resulting device. The cuff 10 may be formed as one continuous channel around the outer edge 20 of the device or into individual segmented compartments 16.

In one embodiment, the cuff 10 may be adapted to prevent cell ingrowth, or alternatively promote cell ingrowth in designated areas.

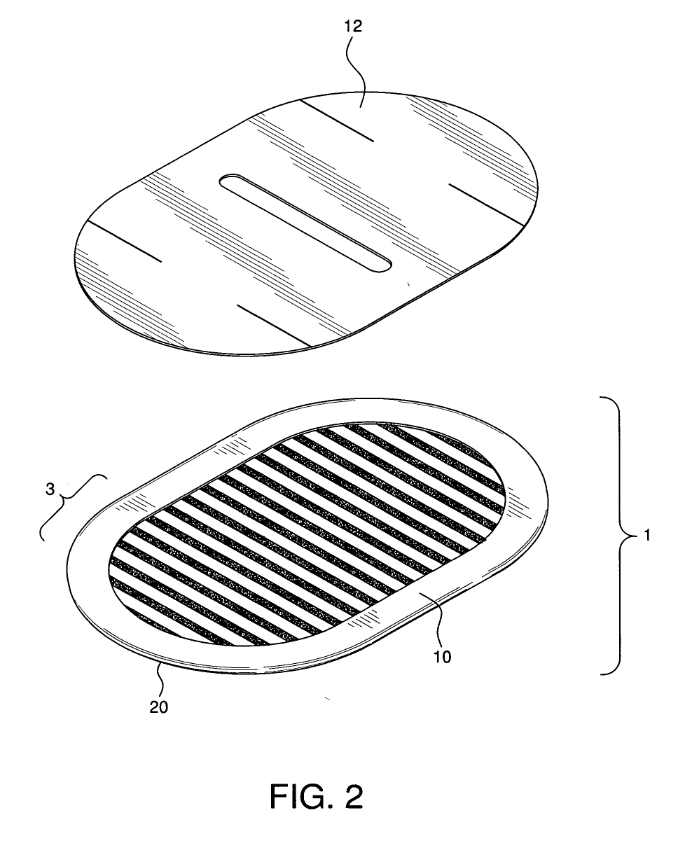

As shown in FIG. 2, a removable support member 12 is of a suitable configuration to be inserted into the implantable medical device 1, so that it is seated between the top side of the tissue repair material 3 and the cuff 10. It is desirable for the removable support member to extend to the outer edge of the device to ensure a sufficient deployment of the device during implantation. The removable support member allows the patch to be easily positioned during implantation. Further, the removable support member allows the patch to remain open during manipulation, and promotes easy guided tack fixation and improved suture placement. The removable support member is oriented between the top side and the cuff 10. The cuff 10 covers secured tacks, protecting or preventing exposure to the surrounding tissue. In one aspect of the invention, the removable support member 12 provides access to the outer edge 20 of the device 1 for suturing or joining to tissues of a patient. The removable support member provides resistant shield to prevent puncture of the underlying tissues during tacking or stapling of the device in place.

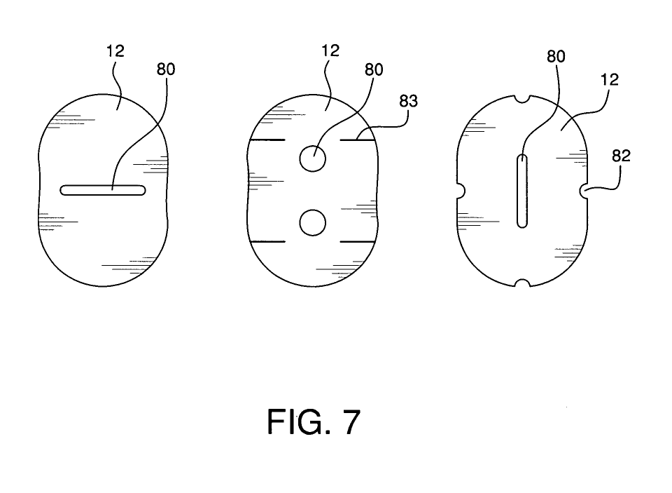

FIGS. 3A, 3B and 3C illustrate retention of the removable support member 12 by the cuff 10. A grip 13 may be present on the removable support member in the form of a cut-out, handle or other suitable aid for manipulation of the removable support during implantation procedures. As shown in FIG. 7, various removable support member 12 designs are contemplated by the present invention. The removable support member 12 can have a continuous boundary, a slit boundary, a boundary with relief cuts as well as multiple grip 13 configurations.

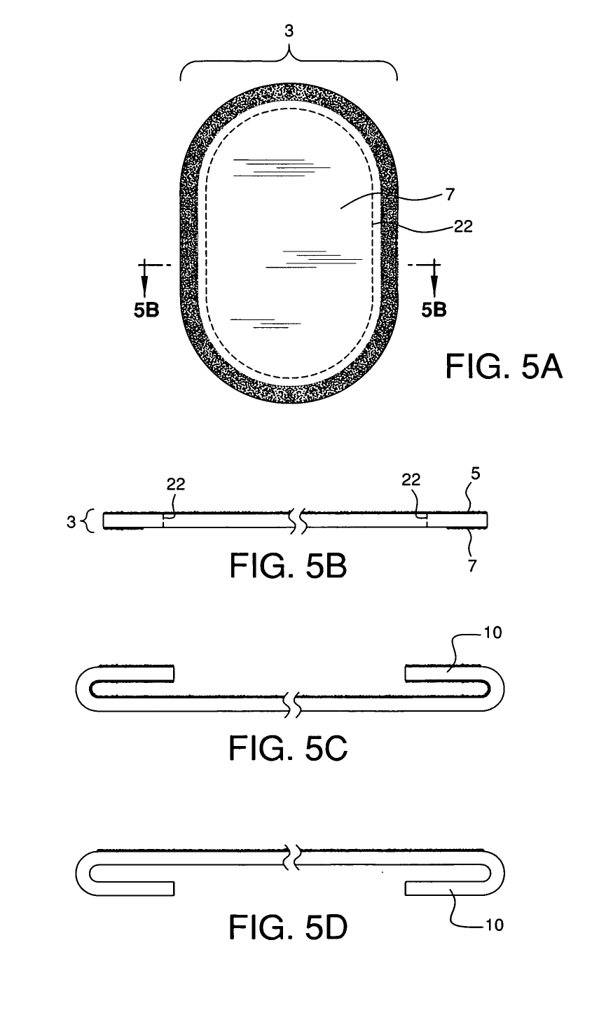

FIG. 5A illustrates a bottom planar view of the bottom side 7 of the tissue repair material 3 prior to cuff formation showing a fold line 22 which creates the overlapped cuff. It may be desirable to incorporate at least one section of bottom surface area that has been roughened, leaving other sections of the tissue repair material 3 surfaces smooth to promote variable cellular ingrowth. FIG. 5B illustrates a cross-sectional view of the mesh prior to cuff formation. In this embodiment, both the top side 5 and bottom side 7 surface areas have been roughened. Only the end portions of the tissue repair material 3 on the bottom side have been roughened in this view, so that when formed into a cuff 10 at fold line 22 as shown in FIG. 5C, the roughened areas all are present on the same side of the device. It should be noted that the device may be oriented in different positions depending upon the desired use, and insertion method. One of the advantages of a smooth surface which is fully continuous, as shown here across the bottom side of the device, is that a continuous barrier property may be achieved across the entire bottom side of the device. The barrier property is useful to prevent adhesions. Particularly useful barriers include ePTFE and PTFE.

While the device of the present invention is particularly suited for open repairs, FIG. 5D shows one configuration which would be particularly suited for laproscopic insertion, as well.

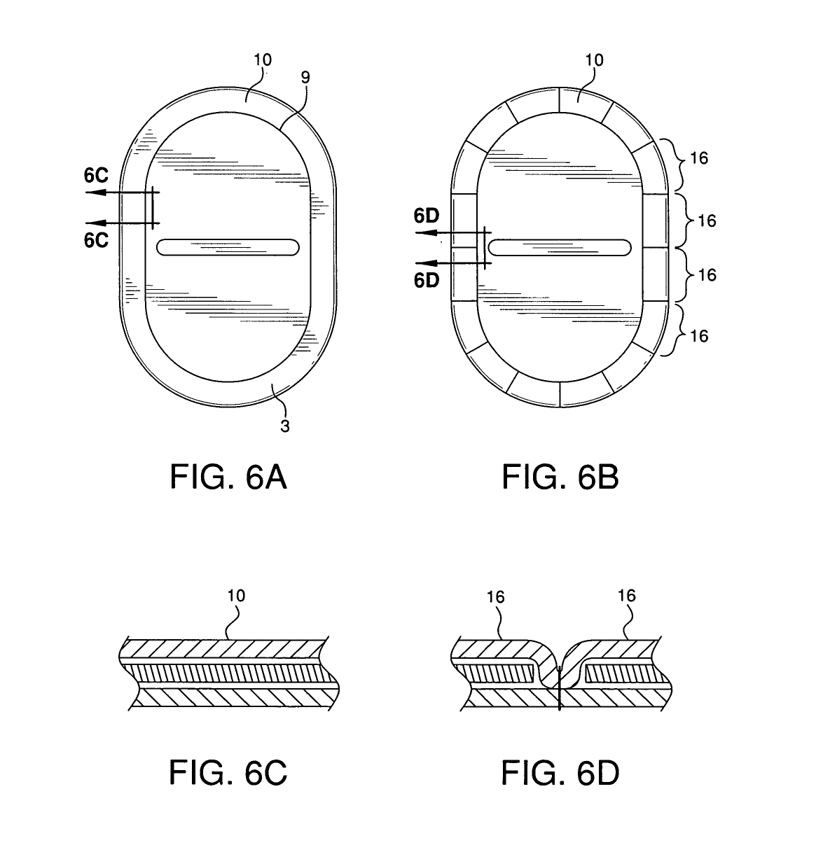

FIGS. 6A and 6B illustrate a top plan view of the present invention with alternative cuff 10 forms. FIG. 6A has a cuff 10 formed as a continuous channel with the opening to the cuff 10 located about the outer perimeter 9 of the tissue repair material 3. FIG. 6B shows a segmented cuff 10 formed as individual segmented compartments 16. The openings to the individual segmented compartments are about the outer perimeter 9 of the tissue repair material 3. FIG. 6C illustrates a cross-sectional view of the mesh showing a continuous cuff 10. FIG. 6D illustrates a cross-sectional view of the mesh showing a non-continuous cuff 10, with individual segmented compartments 16. In both FIGS. 6C and 6D, the removable support member 12 is configured to fit into the cuff 10 for easy use of the device.

One of the many advantages of the present invention is that the removable support 12 may be easily and delicately grasped and removed once the implant is in place. As shown in FIGS. 6, 7, 10 and 12, a surgeon may grasp the removable support member 12 via openings 80 or holes using either his or her fingers or a surgical tool such as forceps. Insertion of the fingers or a tool into a fitted pocket does not necessitate delicate removal of the finger or tool from the fitted pocket once the implant is in place, as other devices require. Further, other devices which require insertion of the surgeon's fingers or a tool into a fitted pocket make delicate removal of the finger or tool from the fitted pocket difficult once the implant is in place.

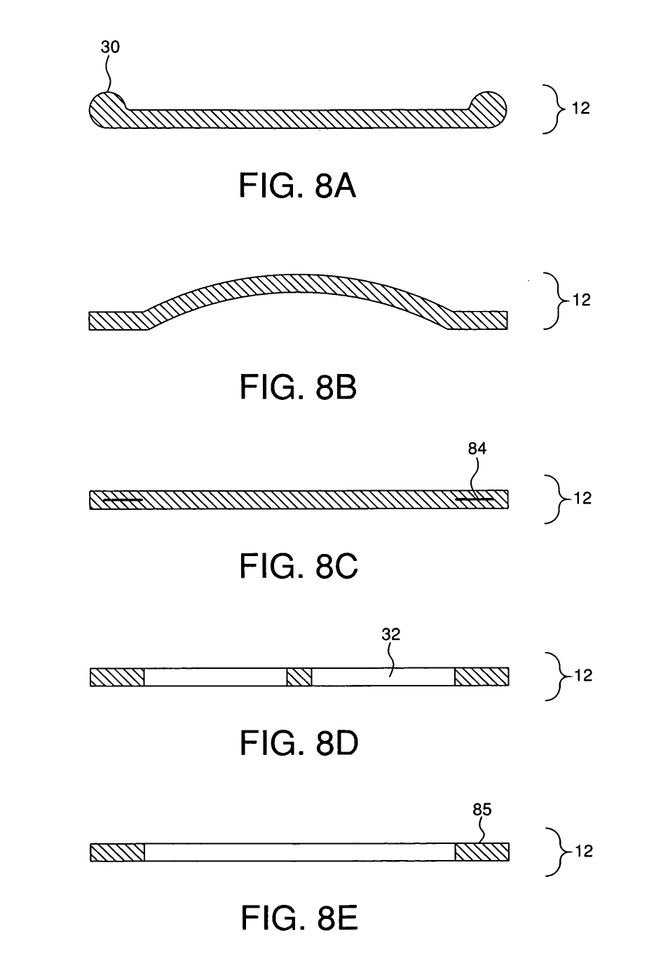

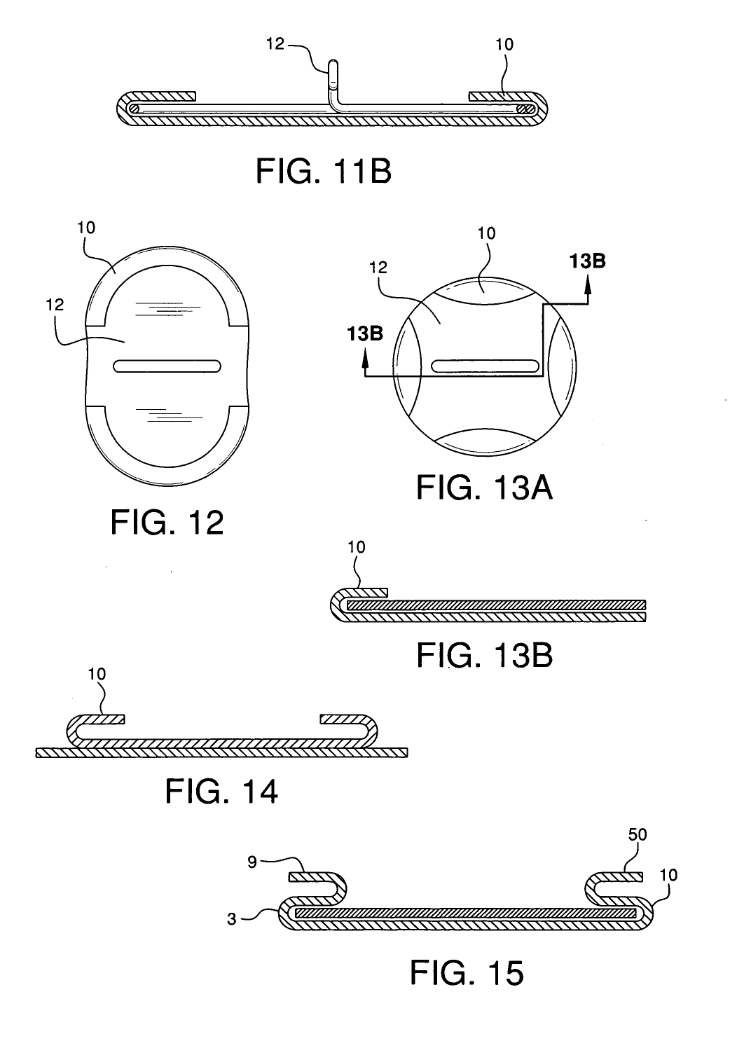

FIGS. 8A through 8E show cross-sectional views of contemplated variations of the removable support member. The removable support member 12 may comprise one or more variations including but not limited to an edge reinforcement 30; a non-planar configuration (FIG. 8B); internal reinforcement such as a reinforcing ring 84 (FIG. 8C); a non-continuous reinforcment configuration 32 (FIG. 8D), an open centered configuration such as a peripheral ring 85 depicted in FIG. 8E. FIG. 11A illustrates a top view of an alternative removable support member 12 which is able to be quickly removed by the application of a force parallel to the support member. FIG. 11B illustrates a cross-sectional view of a removable support member 12 with an upward force required for removal from the cuff 10.

Other variations are contemplated but not depicted.

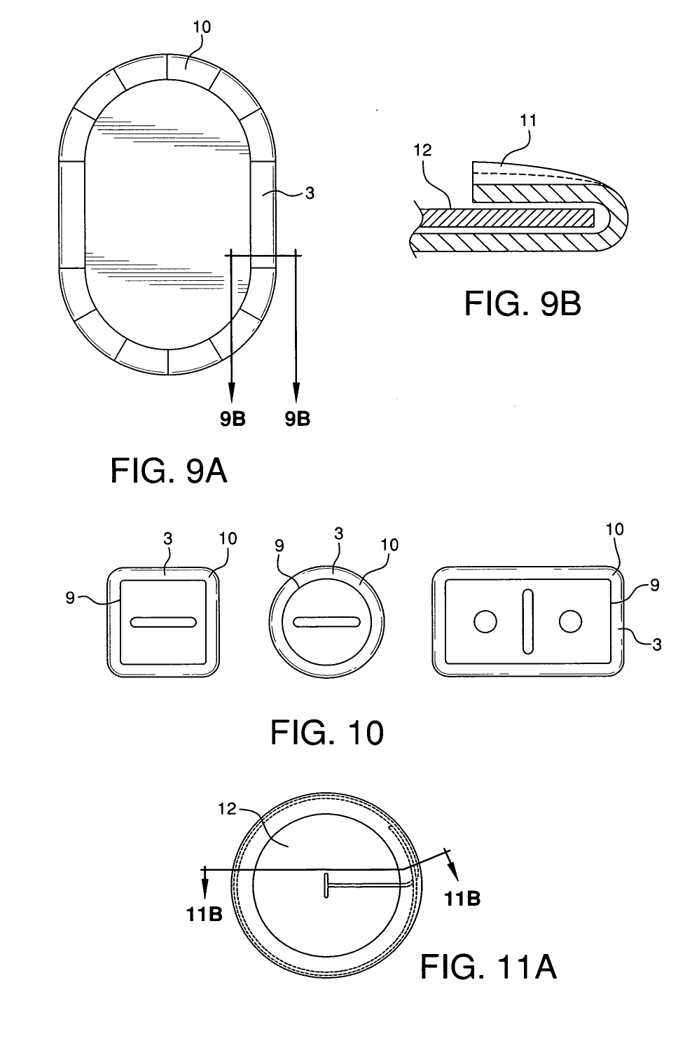

FIG. 9A shows a cuff 10 formed by gathering a planar sheet of tissue repair material 3 so that the individual fold 11 may be pleated and affixed to accommodate the shape of a desired removable support 12. FIG. 9B shows the pleated fold 11 affixed to allow retention of the removable support member 12. FIG. 10 illustrates a top plan view of various shapes and configurations of the present invention wherein the tissue repair material 3 sheet is covered by the continuous overlapping cuff 10. The continuous cuff 10 need only be sized to secure the removable support during placement of the device. The percentage of the tissue repair material 3 sheet which remains uncovered and free of the overlapping continuous cuff 10 formed by the outer perimeter 9 overlapping onto a side of the tissue repair material 3 may range between 5 percent to 95 percent depending upon the configuration of the cuff 10 and the device, but a typical device greater than half of the sheet is exposed. In one embodiment as shown in FIG. 10, more than sixty percent of the tissue repair material 3 sheet remains exposed and separate from the continuous cuff 10. In this example about 40 percent of the tissue repair material 3 sheet is covered by the continuous overlapping cuff 10. The implantable medical device 1 may be comprised of a tissue repair material 3 sheet having two sides and an outer perimeter 9 with at least one side adapted for ingrowth of cells. The cuff 10 may be formed from the outer perimeter 9 to overlap onto a side of the tissue repair material 3 allowing a desired amount of a side adapted for ingrowth of cells of the material sheet exposed. It is desirable in certain configurations such as shown in FIGS. 6, 7, 10, and 12 to have greater than half of the tissue repair material 3 sheet exposed and outside of the continuous overlapping cuff 10.

An implantable medical device 1 comprising a tissue repair material 3 sheet having two sides and an outer perimeter 9 with at least one side adapted for ingrowth of cells, and a cuff 10 formed from portions of the outer perimeter 9 overlapped onto a side of the tissue repair material 3 so that a discontinuous cuff 10 is formed on one side of the tissue repair material 3. The removable support member wherein the tissue repair material 3 is folded over at least a portion of said removable support member.

FIG. 12 illustrates a top plan view of a non-continuous cuff 10 comprising two segments. FIG. 13A illustrates a top plan view of a non-continuous cuff 10 comprising four segments. FIG. 13B illustrates a cross-sectional view of a non-continuous cuff 10 showing how the segment retains the removable support member. As is clear by FIGS. 12 and 13A, the support member may comprise a solid planar removable support member or may comprise a support member which is of relatively open weave such that points of securement occur at some or all of the non-continuous cuff 10 segments.

As shown in FIG. 14, the device may be formed of multiple planar layers so that the cuff 10 may be formed of one or more of the layers, as shown by the cross-sectional view of a two-layer mesh material with the bottom layer extending beyond the cuff 10. This type of an application may be advantageous for securing the patch over or around both symmetrical and non-symmetrical incisions.

In another embodiment, as shown in FIG. 15, the implantable medical device 1 may comprise a tissue repair material 3 having two sides and an outer perimeter, formed so that a first cuff 10 from the outer perimeter 9 of the tissue repair material 3 overlaps onto a side of the tissue repair material 3 and is folded back upon itself forming another cuff (hereinafter referred to as a second cuff 50 for clarity of the invention) from the inner perimeter of the first cuff. The second cuff 50 overlaps onto a side of the first cuff. In one aspect of this embodiment, tissue adjacent to the site of incision may be enclosed between the first cuff and the overlapping flap of the second cuff. This configuration allows for securement through second cuff, the enclosed tissue and the first cuff. The removable support member 12 may then be dislodged leaving the patch firmly secured.

The implantable medical device 1 may be easily placed by attaching one or more suture threads to the perimeter of the device; inserting the surgically implantable device through an incision in the patient; opening and positioning the surgically implantable device such that the device extends beyond the perimeter of the defect; affixing the device to surrounding tissue via the one or more suture threads; and securing the device about the outer perimeter 9 of the device so as to attach the surgically implantable device to the patient. The device may be secured about the outer perimeter 9 using surgical tacks, surgical staples, and surgical sutures. The surgically implantable device may be inserted during an open procedure or laproscopically.

The implantable medical device 1 may be placed by various anchoring mechanisms including: attaching one or more suture threads to the perimeter of the device; inserting the surgically implantable device through an incision in the patient; opening and positioning the surgically implantable device such that the device extends beyond the perimeter of the defect; affixing the device to surrounding tissue via the one or more suture threads; securing the device about the outer perimeter 9 of the device so as to attach the surgically implantable device to the patient; and removing the support member.

The following examples are provided to further illustrate the present invention. These examples are provided to illustrate certain aspects of the invention and are not intended to limit the scope of the invention.

### EXAMPLES

### Example 1

This example describes the construction of a preferred embodiment of the present invention. Following the formation of a support member and the formation of a cuffed implantable sheet material, the support member is fit together with the implantable sheet material such that the formed cuff retains the support member as shown in FIG. 1.

The implantable sheet has a peripheral cuff formed from a continuous implantable sheet such that is folded back onto itself along the entire perimeter between 10.0 mm and 12.0 mm. The implantable sheet was an expanded polytetrafluoroethylene (ePTFE) trade named GORE-TEX® DUALMESH® Biomaterial supplied from the Medical Products Division of W. L. Gore & Associates, Inc. (Flagstaff, Ariz.). The GORE-TEX® DUALMESH® Biomaterial product has a different texture on each side of the sheet. One side is designed to prevent or limit tissue adhesions or other tissue attachments thereto. The other side is roughened to encourage tissue attachment or ingrowth of cells or cellular process therewithin. The GORE-TEX® DUALMESH® Biomaterial was oriented so that the roughened “tissue ingrowth” side is oriented toward the support member.

The cuff was formed by taking the flat implantable sheet as shown in FIG. 5A and placing a 10 cm×15 cm oval folding template on the smooth tissue adhesion barrier side of the implantable sheet and folding the remaining material of the implantable sheet over the edge of the folding plate. The folding plate was formed from a 0.030″ polycarbonate sheet, part number 85585K17, available from McMaster Carr®, Atlanta, Ga. The slack of the folded cuff was taken up into sixteen evenly distributed pleats on each end of the oval, as shown in FIG. 9A and temporarily secured by compressing the pleat using smooth faced pliers, part number 5157A42, available from McMaster Carr®, Atlanta, Ga. The folding plate was removed and as shown in FIG. 9B, these pleats were then sewn at each of their bases using a Series LK-1850 bar tacking sewing machine, available from Juki Corporation, Tokyo, Japan, using CV-5 suture material, supplied from the Medical Products Division of W. L. Gore & Associates, Inc. (Flagstaff, Ariz.). The formed cuffed implantable sheet was then inverted so the cuff was opposite of the smooth tissue adhesion barrier side.

The support member is formed from a continuous sheet of 0.015″ stiff, flexible, resilient material so that when it is bent it wants to return to a flat shape without plastic deformation. The support member has a slit cut into the middle of the sheet lengthwise and ¼″ semi-circular relief cut out in each quadrant, as shown in FIG. 7. The slit is to allow access for positioning during use and the semi-circular relief cut outs are to allow for suture placement without interference with the support member.

The support member is cut from a flat sheet of polycarbonate film, part number 85585K14, available from McMaster Carr®, Atlanta, Ga., using a CO₂ laser, model SL4200 laser, available from Preco, Inc., Lenexa, Kans.

The support member is then fit together with the cuffed implantable sheet so that the support member is constrained by the cuff, as shown in FIG. 3B. The combined components were placed into a heated platen press under 30 psi, at 100° C. under for 30 seconds to flatten and conform the cuffed implantable sheet to the support member.

While particular embodiments of the present invention have been illustrated and described herein, the present invention should not be limited to such illustrations and descriptions. It should be apparent that changes and modifications may be incorporated and embodied as part of the present invention within the scope of the following claims.

The invention claimed is:

1. An implantable medical device comprising:
a. a tissue repair material having two sides and an outer perimeter with at least one side adapted for ingrowth of cells; and
b. a cuff formed from the outer perimeter to overlap onto a side of the tissue repair material.

2. The device of claim 1 further comprising a removable support member wherein the tissue repair material is folded over at least a portion of said removable support member.

3. The implantable medical device of claim 2 wherein the tissue repair material is folded over the support member to form a continuous border which extends around the perimeter of the support member.

4. The implantable medical device of claim 3 wherein the continuous border comprises individual pockets.

5. The implantable medical device of claim 2 wherein the support member is resistant to puncture from fixation device.

6. The device of claim 1 wherein the tissue repair material is conformable.

7. The device of claim 1 wherein the cuff is continuous with and integral to the tissue repair material.

8. The device of claim 1 wherein the cuff and tissue repair material are formed of substantially the same materials.

9. The device of claim 1 wherein the cuff and tissue repair material are formed of substantially different materials.

10. The device of claim 1 wherein tissue repair material is a single material composition.

11. The device of claim 1 wherein tissue repair material comprises expanded polytetrafluoroethylene.

12. The device of claim 1 wherein tissue repair material is a composite of two or more materials.

13. The device of claim 11 wherein tissue repair material is expanded polytetrafluoroethylene.

14. The device of claim 1 wherein tissue repair material comprises at least one bioabsorbable material.

15. The device of claim 1 wherein the cuff has individual segmented compartments.

16. The device of claim 1 wherein the tissue repair material has one or more sides adapted for ingrowth.

17. An implantable medical device comprising:
a. a tissue repair material having two sides and an outer perimeter with at least one side adapted for ingrowth of cells; and
b. a cuff formed from the outer perimeter to overlap onto a side of the tissue repair material wherein cuff provides for fixation at the outer perimeter of the device.

18. An implantable medical device comprising:
a. a tissue repair material having two sides and an outer perimeter;
b. a first cuff formed from the outer perimeter of the tissue repair material to overlap onto a side of the tissue repair material; and
c. a second cuff formed from the inner perimeter of the first cuff to overlap onto a side of the first cuff.

19. The implantable medical device of claim 2 wherein the tissue repair material is affixed via a pressure sensitive adhesive to at least a portion of the removable support member.

20. An implantable medical device comprising:
a. a tissue repair material having a top and bottom side; and
b. a removable support member having a top side, a bottom side, and an outer perimeter, and oriented so that the tissue repair material is in contact with the bottom side of the support member and also in contact with at least a portion of the top side of the support member to form a continuous cuff which extends around and encloses the perimeter of the support member.

21. An implantable medical device comprising:
a. a tissue repair material having two sides and an outer perimeter with at least one side adapted for ingrowth of cells; and
b. a cuff adapted for cell ingrowth formed from the outer perimeter to overlap onto a side of the tissue repair material.

22. An implantable medical device comprising:
a. a tissue repair material having two sides and an outer perimeter with at least one side adapted for ingrowth of cells; and
b. a cuff adapted to prevent cell ingrowth formed from the outer perimeter to overlap onto a side of the tissue repair material.

23. An implantable medical device comprising:
a. a tissue repair material having two sides and an outer perimeter with at least one side adapted for ingrowth of cells; and
b. a cuff adapted to promote cell ingrowth in designated areas formed from the outer perimeter to overlap onto a side of the tissue repair material.

24. A method of placement of an implantable medical device of claim 1 comprising the steps of:
a. attaching one or more suture threads to the perimeter of the device;
b. inserting the surgically implantable device through an incision in the patient;
c. opening and positioning the surgically implantable device such that the device extends beyond the perimeter of the defect;
d. affixing the device to surrounding tissue via the one or more suture threads; and
e. securing the device about the outer perimeter of the device so as to attach the surgically implantable device to the patient.

25. The method according to claim 24, wherein the device is secured about the outer perimeter using surgical tacks.

26. The method according to claim 24, wherein the device is secured about the outer perimeter using surgical staples.

27. The method according to claim 24, wherein the device is secured about the outer perimeter using surgical sutures.

28. The method according to claim 24, wherein the surgically implantable device is inserted laproscopically.

29. A method of placement of an implantable medical device of claim 2 comprising the steps of:
a. attaching one or more suture threads to the perimeter of the device;
b. inserting the surgically implantable device through an incision in the patient;
c. opening and positioning the surgically implantable device such that the device extends beyond the perimeter of the defect;
d. affixing the device to surrounding tissue via the one or more suture threads;
e. securing the device about the outer perimeter of the device so as to attach the surgically implantable device to the patient; and
f. removing the support member.

30. An implantable medical device comprising:
a. a tissue repair material sheet having two sides and an outer perimeter with at least one side adapted for ingrowth of cells; and
b. a cuff formed from the outer perimeter to overlap onto a side of the tissue repair material so that less than 40 percent of the tissue repair material sheet is covered by the continuous overlapping cuff.

31. An implantable medical device comprising:
a. a tissue repair material sheet having two sides and an outer perimeter with at least one side adapted for ingrowth of cells, and
b. a cuff formed from portions of the outer perimeter overlapped onto a side of the tissue repair material so that a discontinuous cuff is formed on one side of the tissue repair material.
